(19) United States
(12) Patent Application Publication
(10) Pub. No.: US 2006/0259002 A1
(43) Pub. Date: Nov. 16, 2006

(54) SANITARY NAPKIN WITH PEAKED ABSORPTION ZONE

(76) Inventor: Delores Parr, Brown Deer, WI (US)

Correspondence Address: GEHRKE & ASSOCIATES, S.C., 123 N. 86th ST, WAUWATOSA, WI, 53226, US

(21) Appl. No.: 10/908,391

(22) Filed: May 10, 2005

(51) Int. Cl. A61F 13/15

(52) U.S. Cl. 604/385.17

(57) ABSTRACT

An anatomically, generally self-conforming sanitary napkin is configured for partially occluding the cavity with respect to fluid flowing therefrom. The sanitary napkin includes a liquid impermeable baffle, a fluid absorbent peaked projection and a cover. The fluid absorbent peaked projection is positioned on the baffle, and includes a raised profile traversing substantially the length of the baffle for partial insertion into a vaginal cavity and between the buttocks of a user. A shape sustaining absorbent core inserted within the peaked projection maintains the raised profile. A liquid permeable hydrous cover is positioned over the absorbent peaked projection, and the cover maintains a moisture level above the fluid absorbent peaked projection.

### BACKGROUND OF THE INVENTION

The present invention relates generally to the field of sanitary napkins for personal feminine care or protection in order to absorb or otherwise contain menstrual fluids or similar exudates. In particular, the present invention relates to a feminine hygiene napkin with a peaked absorption zone traversing the substantial length of the napkin and anatomically contoured to fit between the buttocks and within the vaginal canal to yield a highly efficient catamenial device.

There are many devices configured for the absorption of various body fluids such as menses. These articles should all have certain attributes including absorbency and comfort. In addition to reliably performing a function, these articles should allow a user to be discreet about their use. As is well known, there are two primary articles that attempt to fulfill these objectives: sanitary napkins and tampons.

Sanitary napkins have been developed for external wear about the vulvar region of a user while tampons have been developed for residence within the vaginal cavity. Hybrid devices attempting to merge the structural features of both have been proposed, but have not been very successful primarily because their designs have not found a balance between comfort and functionality.

In particular, some of these devices are designed to project a member through the vaginal orifice. Unlike a tampon which resides entirely within the vaginal canal, this type of device maintains constant interposition within the musculature of the vaginal orifice which is extremely uncomfortable.

Devices for partial or complete interposition within the vestibule of a wearer have failed to achieve any measure of success for a variety of reasons. Many have been designed using projections for disposition within the vestibule but without overall regard for anatomical differences among users, thereby significantly contributing to irritation. Attempt to then secure the article to the undergarment of a wearer creates a rubbing or chafing effect for the user. The design of the article itself is very important in deterring rearward seepage of menses, especially when the user is in a lying or supine position.

Accordingly, those skilled in the art will appreciate that there has yet to be designed a sanitary pad that is specifically anatomically designed to achieve the goals set forth above (e.g., maximize comfort) relative to a contoured sanitary pad but which will overcome the serious disadvantages contributing to their lack of technical and commercial success (e.g, the inability to deter rearward seepage of menses).

### SUMMARY OF THE INVENTION

It is an object of the present invention to provide an anatomically, generally self-conforming sanitary napkin configured for partially occluding the cavity with respect to fluid flowing therefrom. The sanitary napkin includes a liquid impermeable baffle, a peaked projection and a cover. The fluid absorbent peaked projection is positioned on the baffle, and includes a raised profile traversing substantially the length of the baffle for partial insertion into a vaginal cavity and between the buttocks of a user. A liquid permeable hydrous cover is positioned over the absorbent peaked projection, and the cover maintains a moisture level above the fluid absorbent peaked projection.

It is yet another object of the present invention to provide an anatomically, generally self-conforming sanitary napkin configured for partially occluding the cavity with respect to fluid flowing therefrom. The sanitary napkin includes a liquid impermeable baffle, a fluid absorbent peaked projection and a cover. The fluid absorbent peaked projection is positioned on the baffle, and includes a raised profile traversing substantially the length of the baffle for partial insertion into a vaginal cavity and between the buttocks a user. A shape sustaining absorbent core molded into the peaked projection maintains the raised profile. A liquid permeable hydrous cover is positioned over the absorbent peaked projection, and the cover maintains a moisture level above the fluid absorbent peaked projection.

Still yet another object of the present invention is to provide an anatomically, generally self-conforming sanitary napkin configured for partially occluding the cavity with respect to fluid flowing therefrom. The sanitary napkin includes a liquid impermeable baffle, a peaked projection and a cover. The fluid absorbent peaked projection is positioned on the baffle, and includes a raised profile traversing substantially the length of the baffle for partial insertion into a vaginal cavity. A shape sustaining absorbent core is molded into the peaked projection to maintain the raised profile. A liquid permeable hydrous cover is positioned over the absorbent peaked projection, and the cover maintains a moisture level above the fluid absorbent peaked projection.

### BRIEF DESCRIPTION OF THE DRAWINGS

A clear understanding of the various advantages and features of the present invention, as well as the construction and operation of conventional components and mechanisms associated with the present invention, will become more readily apparent by referring to the exemplary, and therefore non-limiting, embodiments illustrated in the following drawings which accompany and form a part of this patent specification.

FIG. 1 is a perspective view of a sanitary napkin with a peaked absorption zone according to the preferred embodiment of the present invention;

FIG. 2 is a perspective view of a sanitary napkin with a peaked absorption zone extending a substantial length of the napkin according to the preferred embodiment of the present invention;

FIG. 3 is an end view of a sanitary napkin with a peaked absorption zone according to the preferred embodiment of the present invention;

FIG. 4 is a cross-sectional view along line 4-4 of FIG. 2 of a sanitary napkin with a peaked absorption zone according to the preferred embodiment of the present invention; and

FIG. 5 is a side view of a shape-sustaining structural element according to the preferred embodiment of the present invention.

### DETAILED DESCRIPTION OF THE PREFERRED EMBODIMENTS

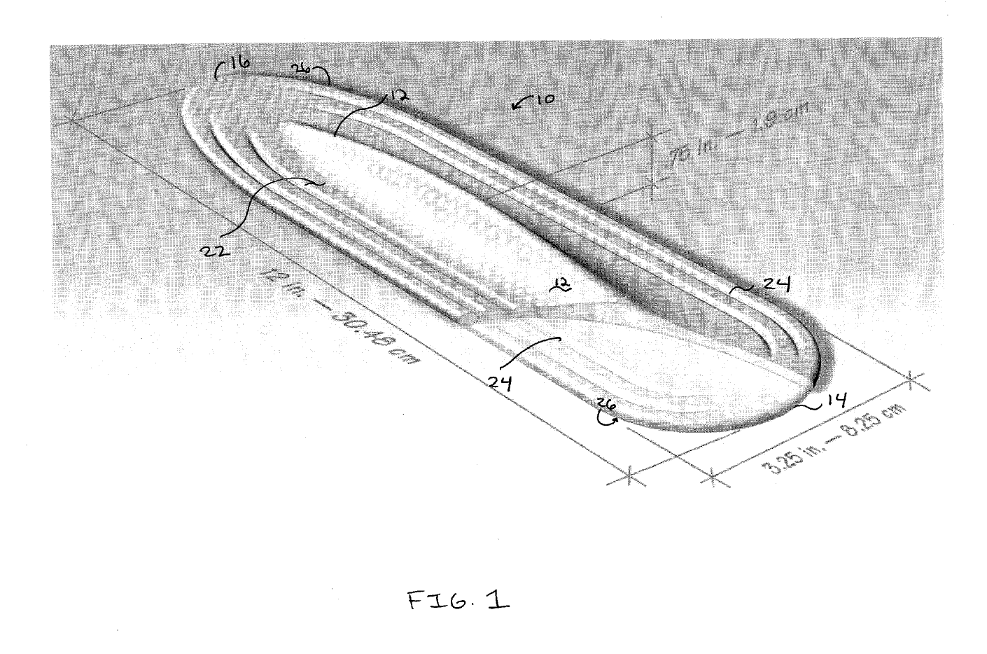

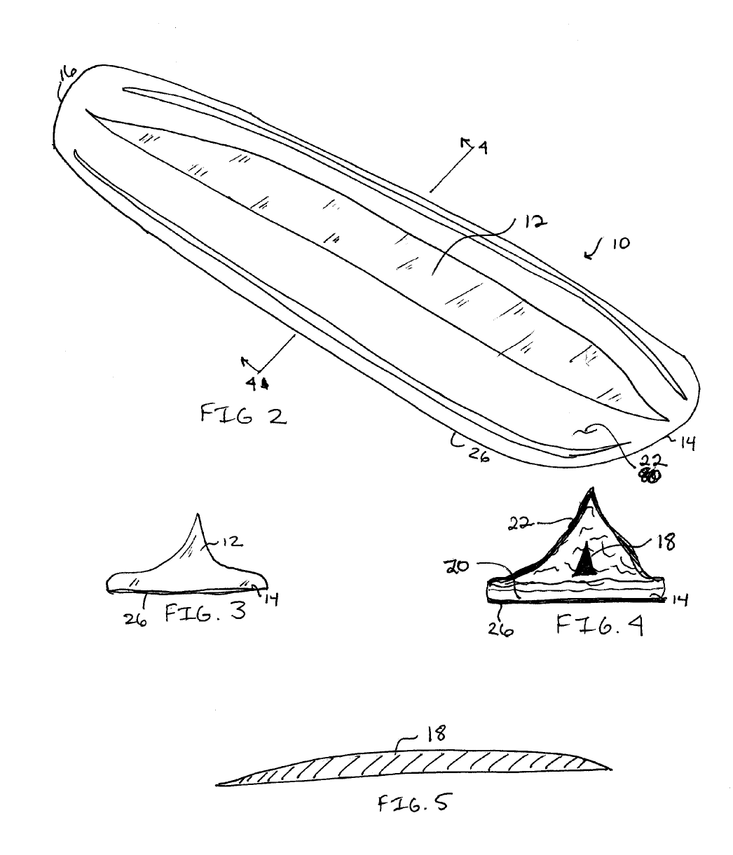

Referring to FIG. 1, the present invention includes generally a sanitary napkin 10 having a peaked projection 12 traversing the substantial length of napkin 10 from a first end 14 to a second end 16. As illustrated in FIG. 2, projection 12 traverses the entire length of napkin 10.

Peaked projection 12 is that region of napkin 10 that protrudes within the vestibule and is contoured to avoid contact with the urogenital elements of the wearer's anatomy. Therefore, the term “projection” conveys the thought that napkin 10 includes a component, preferable integral therewith or otherwise a part thereof, which “projects” upwardly from the external interface or plane of napkin 10 and molds to the particular anatomy of the user.

As illustrated in FIG. 2, peaked projection 12 of napkin 10 is designed to fit between the buttocks and into the vaginal cavity without protruding into the vaginal opening. Peaked projection 12 is the target zone for receipt of menses or like exudates. Those skilled in the art will appreciate that the vast anatomical differences to be encountered among the population of users for these types of devices imply widely varying contours for the target zone and the ability of peaked projection 12 to achieve a self-conforming anatomical cooperation with that side array of potential anatomical variations is a significant advancement.

Napkin 10 is shown in the preferred embodiment to have a generally ovate geometry extending between ends 14 and 16. As illustrated in FIG. 5, peaked projection 12 includes a shape sustaining structural element 18 that is responsible for partially creating the peaked profile. Element 18 only partially maintains the peak because it is important to allow peaked projection 12 to have enough flexibility to conform to the anatomy of the user.

The absorbent capacity of napkin 10 of the present invention is provided by a fluid retentive cord or absorbent body 18. In general, absorbent body 18 is manufactured from conventional absorbent composition including cellulosic batt, individual microfibrous webs, etc.

Alternatively, absorbent body 18 may also be manufactured with other wicking or absorbent materials adapted for retention of fluid including densified celluosic fiber web.

Absorbent body 18 is covered by a cover member 22 which optimally possesses certain characteristics particularly selected on account of the disposition of the pad relative to the user. Rapid Z directional transmission of fluid through the cover and away from the user's body has been the primary concern to those working in this area over the years, along with the necessity of providing a dry cover for the interfacial member which is in proximate engagement with the user's body.

Due to the placement of peaked projection 12 between the user's buttocks and into the vaginal cavity, cover 22 with a conventional dry and absorbing material would cause significant irritation and drying of the delicate tissues and membranes which are physiologically hydrous. This would result in an uncomfortable fit for the user.

Therefore, in the preferred form of the present invention, cover 22 maintains a suitably moist interface so as to not interfere with the moisture level or balance required between the buttocks and into the vaginal cavity. Cover 22 further provides adequate absorbency so as to receive and absorb bodily fluids and conduct them into absorbent body 18.

In operation, bodily fluid is intercepted within peaked projection 12 after it has been emitted from the vaginal orifice. Prior to the time the fluid has an opportunity to migrate rearward as is the case with conventional sanitary pads, the fluid continues to contact peaked projection 12 and distributed for absorption throughout absorbent body 18 within peaked projection 12.

As illustrated in FIG. 1, in the preferred embodiment of napkin 10, end 14 is approximately 3.25 inches wide while end 16 is approximately 3 inches wide which assists a user in determining a back and front of napkin 10. Napkin 10 is approximately 12 inches in length. Napkin 10 further includes an adhesive strip traversing the length of napkin 10 along a napkin back 26 to adhere napkin 10 to undergarments. Back 26 is a liquid impermeable baffle.

Peaked projection 12 is approximately ⅞ inches thick and molded into a peak or pyramid design with a ½ inch to ¾ inch radial slant. The perimeter of projection 12 includes a set of molded channels 24 to wick away any overflow fluid.

The scope of the application is not to be limited by the description of the preferred embodiments described above, but is to be limited solely by the scope of the claims that follow. For example, a conventional cover may be used in place of hydrous cover 22 to wick away fluid towards cord 18 without departing from the scope of the preferred embodiment of the present invention.

What is claimed is:

1. An anatomically, generally self-conforming sanitary napkin configured for partially occluding the cavity with respect to fluid flowing therefrom, the sanitary napkin comprising:
a liquid impermeable baffle;
a fluid absorbent peaked projection positioned on the baffle, wherein the peaked projection includes a raised profile traversing substantially the length of the baffle for partial insertion into a vaginal cavity and between the buttocks of a user; and
a liquid permeable hydrous cover positioned over the absorbent peaked projection, wherein the cover maintains a moisture level above the fluid absorbent peaked projection.

2. The sanitary napkin according to claim 1, wherein the cover comprises nonwoven, spunbounded material.

3. The sanitary napkin according to claim 2, wherein the cover comprises spunweaved polyester.

4. The sanitary napkin according to claim 1, wherein the cover rewicks fluid from the absorbent peaked projection to maintain the moisture level above the fluid absorbent peaked projection.

5. The sanitary napkin according to claim 1, wherein the absorbent peaked projection includes a shape sustaining absorbent core to maintain the raised profile.

6. The sanitary napkin according to claim 5, wherein the peaked projection adapts to the contour of the user's vaginal cavity.

7. An anatomically, generally self-conforming sanitary napkin configured for partially occluding the cavity with respect to fluid flowing therefrom, the sanitary napkin comprising:
a liquid impermeable baffle;
a fluid absorbent peaked projection positioned on the baffle, wherein the peaked projection includes a raised profile traversing substantially the length of the baffle for partial insertion into a vaginal cavity and between the buttocks of a user, and wherein a shape sustaining absorbent core inserted within the peaked projection maintains the raised profile; and
a liquid permeable hydrous cover positioned over the absorbent peaked projection, wherein the cover maintains a moisture level above the fluid absorbent peaked projection.

8. The sanitary napkin according to claim 7, wherein the cover comprises nonwoven, spunbounded material.

9. The sanitary napkin according to claim 8, wherein the cover comprises spunweaved polyester.

10. The sanitary napkin according to claim 7, wherein the cover rewicks fluid from the absorbent peaked projection to maintain the moisture level above the fluid absorbent peaked projection.

11. The sanitary napkin according to claim 7, wherein the peaked projection adapts to the contour of the user's vaginal cavity.

12. An anatomically, generally self-conforming sanitary napkin configured for partially occluding the cavity with respect to fluid flowing therefrom, the sanitary napkin comprising:
a liquid impermeable baffle;
a fluid absorbent peaked projection positioned on the baffle, wherein the peaked projection includes a raised profile traversing substantially the length of the baffle for partial insertion into a vaginal cavity, and wherein a shape sustaining absorbent core molded into the peaked projection maintains the raised profile; and
a liquid permeable hydrous cover positioned over the absorbent peaked projection, wherein the cover maintains a moisture level above the fluid absorbent peaked projection.

13. The sanitary napkin according to claim 12, wherein the peaked projection is inserted between the buttocks of a user.

14. The sanitary napkin according to claim 12, wherein the cover comprises nonwoven, spunbounded material.

15. The sanitary napkin according to claim 12, wherein the cover comprises spunweaved polyester.

16. The sanitary napkin according to claim 12, wherein the cover rewicks fluid from the absorbent peaked projection to maintain the moisture level above the fluid absorbent peaked projection.

17. The sanitary napkin according to claim 12, wherein the peaked projection adapts to the contour of the user's vaginal cavity.

18. The sanitary napkin according to claim 12, wherein the peaked projection forms a geometric pyramid.

19. The sanitary napkin according to claim 12, wherein a channel partially encircling the peaked projection wicks away overflow fluid.
